(19) United States
(12) Patent Application Publication
(10) Pub. No.: US 2026/0204420 A1
(43) Pub. Date: Jul. 16, 2026

(54) FORECASTING OF FUTURE AUTISM DIAGNOSIS AND RESPONSE TO INTERVENTION USING NEURAL DATA AND MACHINE LEARNING

(71) Applicant: The Chinese University of Hong Kong, Shatin, New Territories, Hong Kong SAR (CN)

(72) Inventors: Patrick Chun Man WONG, Tai Po, N.T., Hong Kong SAR (CN); Hoyee Wong HIRAI, Shatin, N.T., Hong Kong SAR (CN); Shaoqi PAN, Shatin, N.T., Hong Kong SAR (CN); Xiujuan GENG, Shatin, N.T., Hong Kong SAR (CN)

(21) Appl. No.: 19/135,342

(22) PCT Filed: Oct. 29, 2024
(86) PCT No.: PCT/CN2024/128122

§ 371 Date: Jun. 3, 2025

Related U.S. Application Data
(60) Provisional application No. 63/594,377, filed on Oct. 30, 2023.

(51) Int. Cl. G16H 50/30; G06F 3/01; G16H 10/60; G16H 20/70; G16H 50/20; G16H 50/70

(52) U.S. Cl. CPC G16H 50/30; G06F 3/015; G16H 10/60; G16H 20/70; G16H 50/20; G16H 50/70

(57) ABSTRACT

A future diagnosis of autism spectrum disorder (ASD) and/or effectiveness of an intervention (such as early intervention) for an individual child can be predicted using a machine-learning model based on neural data such as electroencephalogram (EEG) data, magnetoencephalogram (MEG), and/or magnetic resonance imaging (MRI) data. Training of the model can be based on training data obtained from previous children for whom the intervention was performed.

### CROSS-REFERENCE TO RELATED APPLICATION

This application claims the benefit of U.S. Provisional Application No. 63/594,377, filed Oct. 30, 2023, the disclosure of which is incorporated by reference herein.

### BACKGROUND

The present disclosure relates generally to predicting outcomes in children receiving an intervention related to autism and in particular to using neural data to forecast future diagnosis of autism and response to an intervention.

Autism Spectrum Disorder (ASD) refers to a developmental disability caused by differences in the brain. Globally, the prevalence of ASD has risen drastically in the past decade. For example, in China, prevalence went from 0.39% to 0.7% between 2000 to 2016. In the U.S. the Centers for Disease Control and Prevention (CDC) estimated prevalence in the US at 2.3% in 2021. ASD varies widely in severity and manifestation.

ASD is typically diagnosed based on behavioral symptoms, such as problems with social communication and interaction, restricted or repetitive behaviors, and delays in various developmental milestones. While differences in the brain that give rise to ASD are believed to be present at birth, it is not at present possible to formally diagnose ASD until the child is old enough that behavioral symptoms have become pronounced. Typically, ASD is not diagnosed at ages younger than about four years.

Early intervention (EI), including behavioral intervention, has shown promise in reducing the severity of impact of ASD. For example, in one form of behavioral intervention, parents are coached to implement communicative strategies at home in order to enhance the child's social communication. Clinicians and researchers agree that waiting until age four to begin EI is not optimal, and EI can be prescribed pre-emptively (in advance of an ASD diagnosis) with good results in some cases.

However, EI is not always necessary or effective. When improvement of individual children is examined in detail, a large variability in response to EI can be observed. While some children improve a great deal, others may not improve or may even regress, and improvement or regression cannot be predicted by age or gender of the child. Further, there are different forms of EI, and it is expected that different children will benefit from different interventions.

### SUMMARY

Certain embodiments of the present invention relate to techniques for predicting a future diagnosis of ASD and/or effectiveness of an intervention (such as early intervention) for an individual child. The prediction(s) can be based on neural data such as electroencephalogram (EEG) data, magnetoencephalogram (MEG), and/or magnetic resonance imaging (MRI) data. The neural data can include structural and/or functional data. Quantitative data extracted from the neural data can be provided to a machine-learning model, such as a classifier, that has been trained to predict the effect of a particular intervention. Training can be based on training data obtained from previous children for whom the intervention was performed; the data can include neural data obtained prior to the intervention, a baseline score on an assessment of an ASD-related characteristic of the child administered prior to the intervention, and a second score on the assessment administered after the intervention.

The following detailed description, together with the accompanying drawings, will provide a better understanding of the nature and advantages of the claimed invention.

### BRIEF DESCRIPTION OF THE DRAWINGS

FIG. 1 shows a flow diagram of a process for training a machine-learning model to predict future ASD diagnosis and/or effect of a proposed intervention for an individual child according to an embodiment of the present invention.

FIG. 2 shows a graph of receptive communication scores for individual children before and after an intervention intended to enhance the child's social communication.

FIG. 3 is a flow diagram of a process for predicting an outcome of EI for a child according to an embodiment of the present invention.

FIG. 4 summarizes results obtained from training various machine-learning models according to some embodiments.

FIG. 5 is a circular bar graph illustrating the weight of contributions of neural and non-neural features in predicting the response to intervention using a machine-learning model according to some embodiments.

FIG. 6 is a graph illustrating a correlation between neural EEG data at a first time and scores on ADOS-2 obtained at a later date that can be used according to some embodiments.

FIGS. 7A-7C illustrate examples of correlations between functional connectivity of brain structures and scores on an ASD assessment. FIG. 7A shows brain structures. FIG. 7B shows a graph of ADOS-2 social affect score versus FC of amygdala and inferior frontal cortex for 15 children. FIG. 7C shows a graph of ADOS-2 restricted/repetitive behavior score versus FC of amygdala and posterior Cingulate cortex for 15 children.

### DETAILED DESCRIPTION

The following description of exemplary embodiments of the invention is presented for the purpose of illustration and description. It is not intended to be exhaustive or to limit the claimed invention to the precise form described, and persons skilled in the art will appreciate that many modifications and variations are possible. The embodiments have been chosen and described in order to best explain the principles of the invention and its practical applications to thereby enable others skilled in the art to best make and use the invention in various embodiments and with various modifications as are suited to the particular use contemplated.

### Overview

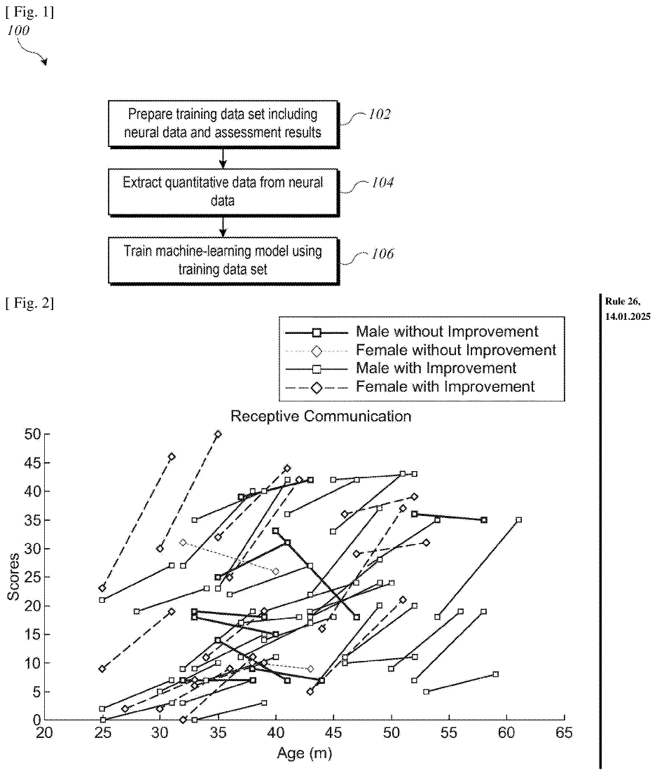

FIG. 1 shows a flow diagram of a process 100 for training a machine-learning model to predict future ASD diagnosis and/or effect of a proposed intervention for an individual child according to an embodiment of the present invention. Process 100 can be implemented using a suitably programmed computer system.

At block 102, a training data set is prepared. The training data set can include information obtained from children who have been diagnosed with ASD and who have undergone an intervention (e.g., early intervention, or “EI”) intended to reduce the severity of ASD. (It should be understood that the ASD diagnosis may have been made before or after the intervention.) The information can include neural data obtained by scanning or monitoring the child's brain prior to the intervention. Examples of neural data include electroencephalogram (EEG) data, magnetoencephalogram (MEG), and/or magnetic resonance imaging (MRI) data. The neural data can include structural an/or functional data. For instance, EEG data, MEG data, and/or MRI data can be recorded while the child is resting and while the child is listening to speech. The neural data can be preprocessed, e.g., to reduce noise, to resample images, to select brain regions of interest, to reduce the size of a data set (e.g., by applying spectral analysis techniques to EEG data), and so on. In some embodiments, the neural data may be obtained from the child prior to a formal ASD diagnosis, e.g., somewhere between birth and about four years of age.

In some embodiments, additional data about the child can also be included. For example, “pediatric” data ca include sex at birth, assessment age, family educational level, family income, and other demographic data. “Psychological” data can include one or more developmental assessment scores determined for the child, e.g., using an assessment tool such as the Autism Diagnostic Observation Schedule, Second Edition (ADOS-2).

The information included in the training data set can also include information quantifying the child's response to the intervention. For instance, behavioral scores for a child can be determined before and after the intervention using an standard instrument that measure the specific ability that the intervention is intended to improve. In one example, the intervention entails parent coaching to improve social communication skills. Communication skills can be measured using a standard assessment tool such as the Vineland Adaptive Behavior Scales. The assessment can be administered before the intervention and after a period of intervention (e.g., six to eight months). The period of intervention can be chosen based on the particular intervention and should be long enough that a response would be expected.

By way of example, FIG. 2 shows a graph of receptive communication assessment scores (determined using the Vineland Adaptive Behavior Scales) for 58 individual children before and after an intervention in which parents were coached to implement communicative strategies at home in order to enhance the child's social communication. The x-axis indicates the child's age (in months) at time of determining the scores, and the y-axis indicates the score; higher scores correspond to higher ability. Scores for the same child before and after the intervention are connected by a line. The child's response can be quantified as the difference in scores. As FIG. 2 shows, there is significant variability in the response to the intervention, with some showing great improvement, some showing small improvement, and a few even regressing. The variability does not correlate well with age, gender, or the initial (pre-intervention) assessment score.

Referring again to FIG. 1, at block 104, quantitative data can be extracted from the neural data. For instance, an EEG may include hundreds or thousands of data samples. Spectral analysis and/or other techniques (such as comparing data sets obtained under different conditions of stimulus or absence thereof) can be used to quantify features of the data set. For MRI, various techniques can be applied to select voxels corresponding to regions of interest and characterize features of such regions using a reduced-size data set.

At block 106, an automated classification algorithm (also referred to as a “classifier” or “machine-learning model”) may be trained using the training data set, including the quantitative data extracted from the neural data. Suitable algorithms include machine-learned classification algorithms such as a Support Vector Machine (SVM), ranking SVM (RankSVM), or random forest.

SVM is a machine-learned classification technique that takes as input a feature vector in a space of arbitrary dimension and a binary classification and maps the feature vector to a point in a classification space such that a hyperplane in the classification space (referred to as a “margin”) separates the points corresponding to the (binary) classification of the corresponding feature vector. In most SVM implementations, the margin may be a “soft margin,” allowing the classification to be less than 100% accurate. In the present context, the feature vector can be the voxel data generated for a given child, and the binary classification can be “low improvement” or “high improvement,” based on the magnitude of the difference between the child's pre-intervention and post-intervention assessment scores.

RankSVM is a machine-learned classification technique whose goal is to construct ordered models that can be used to sort unseen data according to their degree of relevance or importance. RankSVM can be used to form ranking models by minimizing a regularized margin-based pairwise loss. RankSVM uses SVM to compute a weight vector that maximizes the difference of data pairs in ranking. In principle, RankSVM requires investigating every data pair as potential candidates for support vectors, and the number of data pairs is quadratic to the size of the training set. In practice, this can result in low computational efficiency for large training sets and/or large feature vectors. Accordingly, optimizations may be employed to increase computational efficiency; specific examples are described below.

Random forest is a machine-learned prediction or classification technique based on a collection of decision trees. Each decision tree can be trained to make predictions based on a random subset of the features, and different decision trees can be trained using different subsets of the training data. A final prediction can be made by taking an average or mode of the predictions of the decision trees.

Training of a machine-learning model involves automated processes to determine, or “learn,” optimal values for internal parameters of the model, such as the weights for each node or coefficients of a parametric function such as a curve-fitting function or a transform function. A standard approach to training involves iteratively processing data samples through the model and adjusting the parameters of the model, with the goal of minimizing a loss function that characterizes a difference between the output of the model for a given input and an expected result determined from a source other than the model. In embodiments described herein, the expected result for each input training data point can be based on the post-intervention assessment. Loss functions can be selected based in part on the particular model, and optimization of loss functions can proceed using various techniques. Training typically occurs across multiple “epochs,” where each epoch corresponds to a pass through the training sample set. Adjustment to parameters of the model (e.g., weights or coefficients) can occur multiple times during an epoch; for instance, the training data can be divided into “batches” or “mini-batches” and weight adjustment can occur after each batch or mini-batch. Aspects of machine-learning models and training that are relevant to understanding the present disclosure are described herein; any other aspects can be modified as desired.

Referring again to FIG. 1, once the machine-learning model has been trained and validated, the trained machine-learning model can be stored (block 106) for future use with children who are candidates for the intervention.

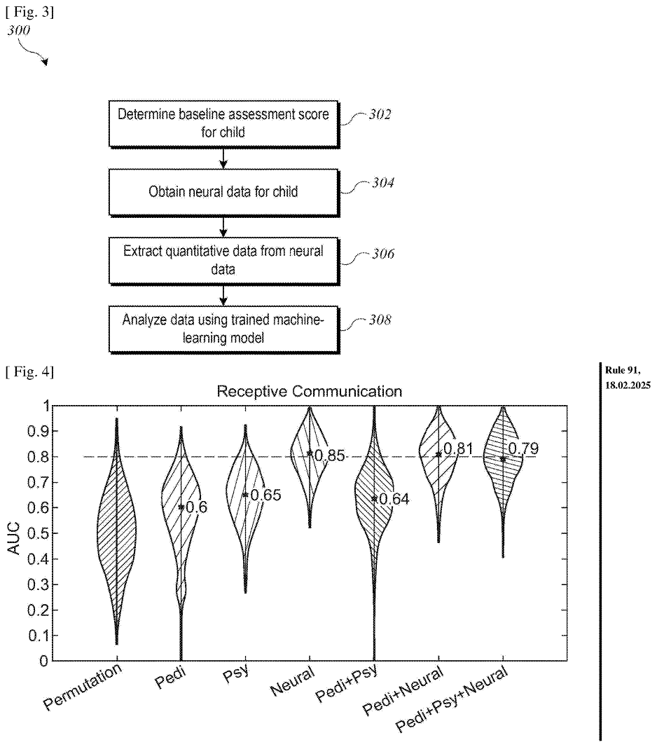

Use of the trained machine-learning model is shown in FIG. 3, which is a flow diagram of a process 300 for predicting an outcome of the intervention for a child according to an embodiment of the present invention. At block 302, a baseline assessment score is determined for the child, e.g., by administering the same assessment that was used for the children included in the training data. At block 304, neural data for the child is obtained. The same techniques and modalities used to obtain the neural data for children in the training data set are used. Other data for the child may also be obtained, including pediatric data and/or psychological data (e.g., scores on other developmental assessment tests). At block 306, quantitative data is extracted from the neural data. The same techniques and modalities used to extract quantitative data from the neural data for children in the training data set are used. At block 308, the data is analyzed using the trained machine-learning model. The machine-learning model applies the trained algorithm and outputs a classification result e.g., a predicted degree of improvement in response to the intervention that was received by children in the training data set. Depending on the particular machine-learning model, the predicted degree of improvement may correspond to a binary “low improvement” or “high improvement” classification or to a quantified degree of improvement. At block 306, the predicted degree of improvement and the child's baseline ASD score can be used to generate a predicted outcome. This predicted outcome can be provided to a clinician for use in treatment planning.

Those skilled in the art will appreciate that a process similar to process 300 can also be used for validation of models during training process 100. A training sample can be input into a model that is being validated to obtain a “predicted” outcome. In this case, the actual outcome is also known, and comparing the “predicted” outcome to the actual outcome provides an indication of accuracy of the model.

In some embodiments, the model can be self-updating. For instance, once con-structed, the model can be used to evaluate new (previously unseen) children who are candidates for the intervention. As children who are evaluated using the model receive intervention and have their outcomes determined, data for these children can be added to the data set used for training, and the model can be updated from time to time (e.g., by repeating processes 100 and 200 using the enlarged data set). The updated model can then be used to predict outcomes for additional children.

In some embodiments, a machine-learning model can be trained to forecast a future ASD diagnosis in the absence of any intervention other than allowing time to pass. For instance, the training data can be obtained from children who did not receive an intervention. Training data can include neural data obtained at a first time, a baseline assessment of an ASD-related characteristic at or near the first time, and a subsequent assessment of the same ASD-related characteristic at a second time, which can be, e.g., several months or a year or more after the first time. Between the first time and the second time, the child can be allowed to develop in their naturalistic environment without receiving any specific intervention aimed at reducing ASD symptoms or behaviors. A machine-learning model trained using this data can predict, or forecast, a future ASD diagnosis in the absence of active intervention. In some embodiments, such a prediction can be used as a “control” and compared to predictions for active interventions.

### EXAMPLES

To further illustrate these processes, specific examples of classification models that have been trained to predict outcomes of EI in an experimental context will now be described. These examples are based data from 58 children who underwent EI in the form of parent coaching as described above. The children were between 25 and 54 months of age at the time of enrollment for the intervention. They had been evaluated by a qualified medical professional and assessed as having either autism or an elevated likelihood of autism requiring close monitoring. Each child was also assessed using ADSOS-2 and scored in the autism or autism spectrum range, and the Vineland Adaptive Behavioral Scales were used to measure the child's receptive communication ability before and after intervention.

Several machine-learning models using a random forest algorithm were trained to predict improvement in receptive communication ability. Different models received different combinations of data. A first model, referred to as “Pedi,” received only pediatric data, including sex at birth, age at assessment, family educational level, and family income. A second model, referred to as “Psy,” received the child's developmental assessment scores, including the ADOS-2 and initial Vineland scores. A third model, referred to as “Neural,” received EEG data including the child's neural encoding of speech (“frequency following response”data) and resting-state (“rest”) EEG data. Additional models received combinations of these data types, including “Pedi+Psy,” “Pedi+Neural,” and “Pedi+Psy+Neural.” Each model was trained multiple times using different random subsets of the training data and validated using the remaining training data.

FIG. 4 summarizes results obtained from training and validating the various machine-learning models. Different models are listed along the x-axis; the y-axis plots the distribution of receiver area under the curve (AUC) for each model. A random permutation is shown for comparison. The stars indicate the median AUC. As FIG. 4 shows, models that include neural data outperform models that do not include neural data. AUC of 0.8 can be considered as a threshold for clinical significance, and only models that include neural data are capable of reaching this threshold.

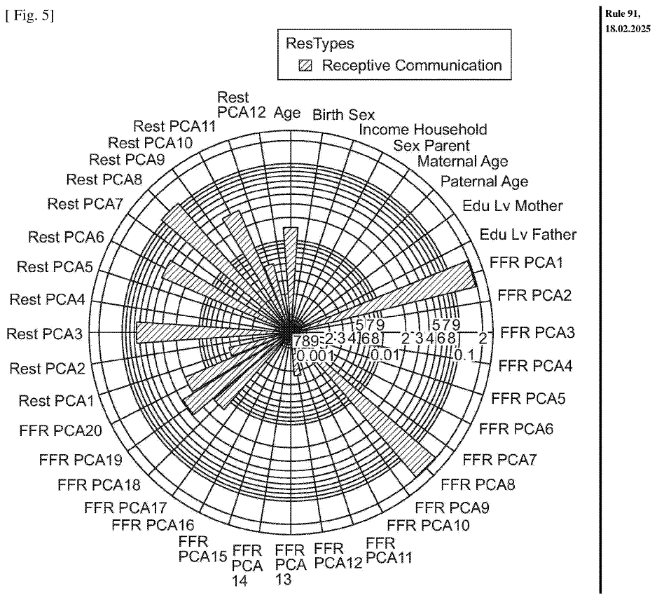

FIG. 5 is a circular bar graph illustrating the weight of contributions of neural and non-neural features in predicting the response to intervention using a machine-learning model according to some embodiments. Each sector of the circle corresponds to a different feature input to the machine-learning model. Neural features are labeled in red (with names beginning with “FFR” or “Rest”) , and non-neural features are labeled in white. The size of the bar corresponds to the weight of each feature. As can be seen, neural features consistently have the highest weights.

### Computer Implementations

Data analysis and computational operations of the kind described herein can be implemented in computer systems that may be of generally conventional design, such as a desktop computer, laptop computer, tablet computer, mobile device (e.g., smart phone), or the like. Such systems may include one or more processors to execute program code (e.g., general-purpose microprocessors usable as a central processing unit (CPU) and/or special-purpose processors such as graphics processors (GPUs) that may provide enhanced parallel-processing capability); memory and other storage devices to store program code and data; user input devices (e.g., keyboards, pointing devices such as a mouse or touchpad, microphones); user output devices (e.g., display devices, speakers, printers); combined input/output devices (e.g., touchscreen displays); signal input/output ports; network communication interfaces (e.g., wired network interfaces such as Ethernet interfaces and/or wireless network communication interfaces such as Wi-Fi); and so on.

Computer programs incorporating features of the present invention that can be implemented using program code may be encoded and stored on various computer readable storage media; suitable media include magnetic disk or tape, optical storage media such as compact disk (CD) or DVD (digital versatile disk), flash memory, and other non-transitory media. (It is understood that “storage” of data is distinct from propagation of data using transitory media such as carrier waves.) Computer readable media encoded with the program code may include an internal storage medium of a compatible electronic device and/or external storage media readable by the electronic device that can execute the code. In some instances, program code can be supplied to the electronic device via Internet download or other transmission paths.

In alternative embodiments, a purpose-built processor may be used to perform some or all of the operations described herein. Such processors may be optimized, e.g., for performing computations to train a random forest or other machine-learning model, and may be incorporated into computer systems of otherwise conventional design or other computer systems.

### Additional Embodiments

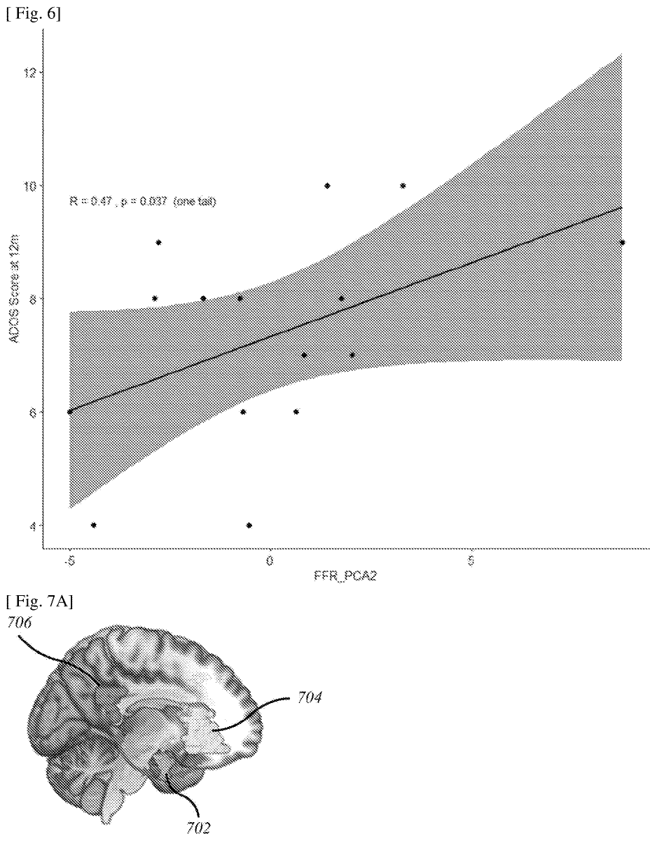

In some embodiments, a machine-learning model of the kind described above can be trained to forecast a future ASD diagnosis. For example, it is observed that neural EEG data at a first time correlates with scores on ADOS-2 obtained at a later date. FIG. 6 illustrates the effect for a particular feature (FFR_PCA2) extracted from EEG data. As shown for a sample of 15 children, FFR_PCA2 correlates with ADOS-2 scores obtained 12 months later (r=0.47, p=0.037 [one tail]). It is expected that other EEG features may also have correlations with future ADOS-2 scores, and training a machine-learning model using multiple EEG features and/or other neural data may enable a predictive diagnosis of autism based on neural data, e.g., before the child is old enough for a conventional diagnosis.

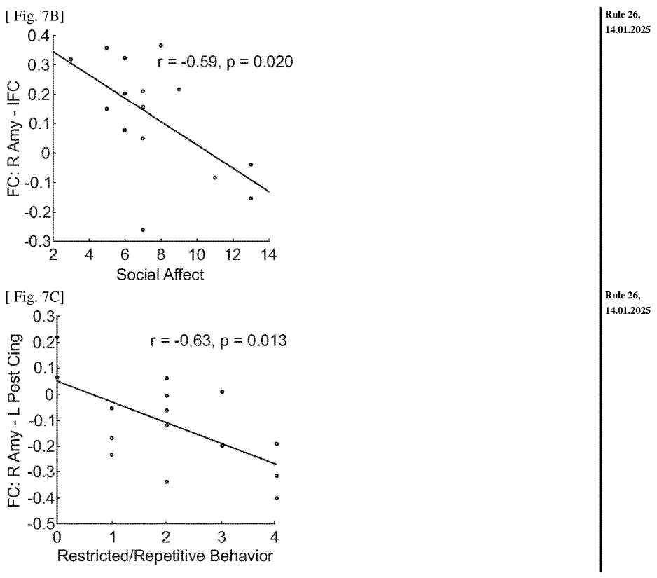

In some embodiments, MRI data can be used in addition to or instead of EEG data to forecast autism severity. For example, it is observed that functional connectivity measures derived from functional MRI data correlate with ASD-related assessment scores. FIG. 7A shows brain structures including the amygdala 702, inferior frontal cortex 704, and posterior Cingulate cortex 706. Functional connectivity (FC) is indicated by blue arrows between the structures. FIG. 7B shows a graph of ADOS-2 social affect score versus FC of amygdala and inferior frontal cortex for 15 children. A correlation is observed (r=−0.59, p=0.020). Similarly, FIG. 7C shows a graph of ADOS-2 restricted/repetitive behavior score versus FC of amygdala and posterior Cingulate cortex for 15 children. A correlation is observed (r=−0.63, p=−0.013). Thus, fMRI data is expected to be useful input for forecasting the effect of EI and/or predicting a future ASD diagnosis.

While the invention has been described with reference to specific embodiments, those skilled in the art will appreciate that variations and modifications are possible. For instance, while examples describe above focus on early intervention to improve communication skills and in particular receptive communication skills, other interventions may target other symptoms of ASD. Using techniques described herein with appropriate assessments to measure the effect of an intervention on an individual basis, effectiveness of other interventions can be predicted.

Neural data can include data characterizing structure and/or function of any portion of the child's central nervous system, including the brain. Structural data can be obtained, e.g., using MRI. Functional data can be collected using EEG, MEG, functional near-infrared spectroscopy (fNIRS), functional magnetic resonance imaging (fMRI), or other modalities. Neural data collected using different modalities an be used in combination or separately. Neural data may include data representing the entire brain or specific regions or structures within the brain or other portions of the central nervous system. In some embodiments, some data can be acquired while the child is sedated or in natural sleep, and some data can be acquired while the child is exposed to a stimulus, such as speech or visual stimulus.

Quantitative data can be extracted from neural data using various techniques. For example, quantitative data can be extracted from EEG data using spectral analysis techniques or the like. For imaging data such as MRI or fMRI data, image registration and masking techniques can be used to select regions of interest, and various image analysis techniques can be applied to extract features from the images, to reduce the size of input feature vectors to the machine-learning model. Data obtained under different conditions (e.g., at rest versus when exposed to stimulus or when exposed to two or more different stimuli) can be compared to determine differences, and the differences can be used to define quantitative data.

Classification of children according to degree of improvement can be based on any measurement of communication skill and/or behavior, including but not limited to the Vineland assessment, ADOS-2, or other tests described above. For a given child, a baseline (pre-EI) assessment can be used as a reference point for the predicted improvement, allowing an overall assessment of likely outcome in terms of post-EI communication skills.

Techniques described herein can be applied to predict the effect of any intervention relevant to autism, including behavioral interventions, neural interventions, pharmacological interventions, and so on. Some interventions may be indirect (e.g., training parents to deliver therapy in the child's naturalistic setting), and some may be direct (e.g., medication administered to the child). In some embodiments, different machine-learning models can be trained to predict the outcomes of different interventions (or different combinations of interventions), using the same input features or different subsets of the input features. A machine-learning model can also be trained using children who were not given any intervention, and such a model can predict a future ASD-related diagnosis for the child in the absence of intervention. For instance, the prediction can pertain to presence or severity of ASD or of any characteristic associated with autism, autism-like behaviors on a spectrum, or autism subtypes. Examples of such characteristics include emotion and self-regulation; social interaction; social communication; language and communication; motor skills; executive function; cognition; and/or restricted or repetitive patterns of behavior, interests, or activities.

A variety of machine-learning algorithms (including any classifier or other algorithm that can be trained to predict an outcome for an unseen data sample based on a set of data samples with known outcomes) can be used. In examples described above, a random forest model can be used to quantitatively predict improvement as a result of EI. Other models can be substituted, such as Support Vector Regression (SVR), Hidden Markov Models, Bayesian classifiers, classifiers based on univariate or multivariate analytics, and deep learning algorithms (e.g., artificial neural networks). In various embodiments, predictions may be quantitative or qualitative. For example, SVM can provide a binary classifier that can be used to indicate whether the child is likely or unlikely to experience significant improvement as a result of EI. (For instance, a minimum difference between assessment scores after and before intervention can be can be defined as a threshold for significant improvement.) Where improvement is measured on an ordinal scale (e.g., high, moderate, low, none), a RankSVM or similar model may allow a prediction of the level of improvement. Other models can output numerical predictions, e.g., a quantitative amount of predicted improvement or a probability of significant improvement, or the like. The parameters used for training and testing the model may be varied, including the size of training data sets and the particular combination of input features. A particular algorithmic implementation of training is not required.

The training data set can encompass children over a range of ages, e.g., from birth to 4 years or from birth to 12 years or the like.

Predicted outcomes generated in the manner described herein may be used in treatment planning. For example, the predicted outcome may inform the decision whether to proceed with an intervention for a given child. As another example, where separate machine-learning models can be trained to predict effectiveness of different interventions (or combinations of interventions or no intervention), predictions from a set of models can inform selection of intervention(s) for a given child. For instance, a prediction based on no intervention can be compared to predictions for various interventions.

All processes described herein are also illustrative and can be modified. Operations can be performed in a different order from that described, to the extent that logic permits; operations described above may be omitted or combined; and operations not expressly described above may be added.

While various circuits and components are described herein with reference to particular blocks, it is to be understood that these blocks are defined for convenience of description and are not intended to imply a particular physical arrangement of component parts. The blocks need not correspond to physically distinct components, and the same physical components can be used to implement aspects of multiple blocks. Components described as dedicated or fixed-function circuits can be configured to perform operations by providing a suitable arrangement of circuit components (e.g., logic gates, registers, switches, etc.); automated design tools can be used to generate appropriate arrangements of circuit components implementing operations described herein. Components described as processors or microprocessors can be configured to perform operations described herein by providing suitable program code. Various blocks might or might not be reconfigurable depending on how the initial configuration is obtained. Embodiments of the present invention can be realized in a variety of apparatus including electronic devices implemented using a combination of circuitry and software.

Accordingly, although the invention has been described with respect to specific embodiments, it will be appreciated that the invention is intended to cover all modifications and equivalents within the scope of the following claims.

1. A method of predicting an outcome of an intervention for a child with actual or potential autism spectrum disorder (ASD), the method comprising:
obtaining neural data characterizing one or both of anatomical or functional properties of at least a portion of the child's central nervous system;
extracting quantitative data from the neural data;
determining a baseline assessment of an ASD-related characteristic of the child;
determining a predicted change in the baseline assessment for the child by analyzing the quantitative data using a machine-learning model that has been trained to predict a change in assessment of the ASD-related characteristic of the child, wherein the training of the machine-learning model is based on corresponding quantitative data and assessments of the ASD-related characteristic from a training data set obtained from a plurality of previous children having known outcomes of the intervention; and
generating a predicted outcome of the intervention based on the baseline assessment and the predicted change in the baseline assessment.

2. The method of claim 1 wherein the neural data includes electroencephalogram (EEG) data.

3. The method of claim 1 wherein the neural data includes resting data and data obtained while the child is subject to a communicative stimulus.

4. The method of claim 1 wherein the neural data includes one or more of:
electroencephalogram (EEG) data;
magnetic resonance imaging (MRI) data;
functional magnetic resonance imaging (fMRI) data;
magnetoencephalogram (MEG) data; or
functional near-infrared spectroscopy (fNIRS).

5. The method of claim 1 wherein the quantitative data includes one or more of:
first functional data characterizing a cortical or subcortical response when at rest;
second functional data characterizing a cortical or subcortical response to a first stimulus;
third functional data characterizing a cortical or subcortical response to a second stimulus different from the first stimulus;
data indicating a difference between the first functional data and the second functional data; or
data indicating a difference between the second functional data and the third functional data.

6. The method of claim 1 wherein the quantitative data includes one or more of:
data characterizing one or more of morphology, volume, or thickness of one or more regions of the child's central nervous system;
data characterizing integrity of white matter tracks in one or more regions of the child's central nervous system;
data characterizing functional connectivity between two structures of the child's central nervous system;
data characterizing structures along one or more cortical or subcortical pathways;
data characterizing a function along a neural pathway;
data characterizing a function of a processing center of a sensory neural pathway;
data characterizing a function of a brainstem nucleus and a connected pathway; or
data characterizing a function of a thalamic nucleus and a connected pathway.

7. The method of claim 1 wherein the ASD-related characteristic is a characteristic associated with autism, autism-like behaviors on a spectrum, or autism subtypes.

8. The method of claim 7 wherein the ASD-related characteristic includes one or more of:
emotion and self-regulation;
social interaction;
social communication;
language and communication;
motor skills;
executive function;
cognition; or
restricted or repetitive patterns of behavior, interests, or activities.

9. The method of claim 1 wherein determining the baseline assessment includes administering an assessment tool.

10. The method of claim 9 wherein the assessment tool is administered online or in-person, by a natural person or by a machine.

11. The method of claim 9 wherein the known outcomes of the intervention for the previous children in the training data set are determined by administering the assessment tool a second time after the intervention.

12. The method of claim 1 wherein the intervention includes an early intervention performed in advance of a formal autism diagnosis.

13. The method of claim 1 wherein the intervention includes one or more of:
a behavioral therapy administered by a professional;
a behavioral therapy administered by a parent in a naturalistic setting for the child;
a neural intervention; or
a pharmacological intervention.

14. The method of claim 1 wherein the intervention includes allowing time to pass and the predicted outcome corresponds to a prediction of a future diagnosis of autism or ASD.

15. The method of claim 1 wherein the machine-learning model comprises a random forest model.

16. The method of claim 1 wherein the machine-learning model comprises a support vector machine (SVM).

17. The method of claim 1 wherein the machine-learning model is based on Bayesian inferencing.

18. The method of claim 1 wherein the machine-learning model is based on univariate or multivariate analytics.

19. The method of claim 1 wherein the predicted outcome includes a quantitative prediction of improvement.

20. The method of claim 1 wherein the predicted outcome includes a qualitative prediction of improvement.

21. The method of claim 20 wherein the qualitative prediction is either a significant improvement or not a significant improvement.

22. The method of claim 1 further comprising:
obtaining birth, family, and health data of the child,
wherein the birth, family, and health data of the child is input to the machine-learning model together with the quantitative data.

23. The method of claim 1 wherein a score reflecting the baseline assessment of the ASD-related characteristic of the child is input to the machine-learning model together with the quantitative data.

24. The method of claim 1 further comprising:
obtaining additional psychological data of the child, including a score on one or more additional assessments of ASD-related characteristics,
wherein the additional psychological data is input to the machine-learning model together with the quantitative data.

25. The method of claim 1 wherein the training data set includes neural data for children obtained from birth to 12 years and developmental assessment results obtained at an age of at least 4 months.

26. The method of claim 1 wherein the child is under 12 years of age and the training data was obtained from previous children at ages under 12 years.

27. A system comprising:
a memory; and
a processor coupled to the memory and configured to:
obtain, for a child with actual or potential autism spectrum disorder (ASD), neural data characterizing one or both of anatomical or functional properties of at least a portion of the child's central nervous system;
extract quantitative data from the neural data;
determine a baseline assessment of an ASD-related characteristic of the child;
determine a predicted change in the baseline assessment for the child by analyzing the quantitative data using a machine-learning model that has been trained to predict a change in assessment of the ASD-related characteristic of the child, wherein the training of the machine-learning model is based on corresponding quantitative data and assessments of the ASD-related characteristic from a training data set obtained from a plurality of previous children having known outcomes of an intervention; and
generate a predicted outcome of the intervention based on the baseline assessment and the predicted change in the baseline assessment.
